(19) United States
(12) Patent Application Publication
(10) Pub. No.: US 2015/0157657 A1
(43) Pub. Date: Jun. 11, 2015

(54) PREPARATION AND DELIVERY OF SUSTAINED NITRIC OXIDE RELEASING SOLUTIONS

(71) Applicant: Bovicor Pharmatech Inc., Vancouver (CA)

(72) Inventors: Alex Stenzler, Long Beach, CA (US); Steve Han, Huntington Beach, CA (US); Christopher C. Miller, North Vancouver (CA); Gilly Regev-Shoshani, Vancouver (CA)

(21) Appl. No.: 14/516,930

(22) Filed: Oct. 17, 2014

Related U.S. Application Data
(60) Provisional application No. 61/892,681, filed on Oct. 18, 2013.

(51) Int. Cl. A61K 33/00; A61M 35/00; A61M 15/00; A61M 11/02; A61M 11/00

(52) U.S. Cl. CPC A61K 33/00; A61M 11/02; A61M 11/007; A61M 15/009; A61M 35/00; A61M 2205/50; A61M 2202/0275; A61M 2205/82; A61M 2205/07

(57) ABSTRACT

The present invention relates to a system and method of treating a subject with nitric oxide gas and/or nitric oxide releasing solutions. The present invention also relates to compositions and devices useful for treating a subject with nitric oxide gas and/or nitric oxide releasing solutions.

### PRIORITY DATA

This application claims the benefit of U.S. Provisional Patent Application Ser. No. 61/892,681, filed on Oct. 18, 2013, which is incorporated herein by reference.

### BACKGROUND OF THE INVENTION

It is known that nitric oxide gas has an antimicrobial effect, and when safely administered, can be used as a therapeutic treatment of microbial infection in a subject. While many systems have been described for the use of nitric oxide in clinical settings, these systems are designed for the delivery of nitric oxide gas to the subject, which requires the subject to remain stationary for an extended period of time. Unfortunately, many instances where treatment of nitric oxide would be particularly beneficial do not allow for the subject to be stationary or immobilized for the length of time needed to receive an effective dosage of nitric oxide gas.

For example, one such instance is in the cattle industry, where Bovine Respiratory Disease (BRD) continues to be the most common disease in the feeder beef cattle in North America, effecting 20-40% of receiver calves annually. Production losses from BRD include respiratory morbidity and mortality as well as increased treatment and processing cost. Its pathogenicity has been linked to a primary viral infection followed by a secondary bacterial infection.

The incidence of BRD has been shown to be reduced in animals treated with nitric oxide gas. However, this treatment requires 30 minutes of exposure, which is a very significant amount of time during commercial operations and difficult to implement. Unfortunately, in practice, the calves are placed in holding chutes for less than 5 minutes, a period that is insufficient to deliver an adequate or effective dose of nitric oxide gas. Additionally, while liquids capable of releasing nitric oxide have been demonstrated in experimental settings, there is a need for a device and method that enable production of such a liquid at a cattle feed lot immediately before its use.

It has previously been demonstrated that nitric oxide gas can be delivered to the upper respiratory tract of cattle to reduce the incidence of BRD. Also previously described are formulations of an acidified nitrate solution for the production of gNO used as an antimicrobial agent (U.S. Pat. No. 6,709,681). However, once formulated, the nitric oxide immediately comes off, and so during applications such as the treatment of cattle during commercial applications, too much of the gas would be lost, making such NO producing formulation ineffective.

Accordingly, the present inventors have recognized a need for a device and method to quickly and efficiently deliver a long acting and effective dosage of nitric oxide gas. The present invention satisfies this need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic of an exemplary nitric oxide releasing solution delivery device.

FIG. 2 is a schematic of an exemplary nitric oxide releasing solution delivery device.

FIG. 3 is a schematic of an exemplary nitric oxide releasing solution delivery device.

FIG. 4 is a schematic of an exemplary nitric oxide releasing solution delivery device.

FIG. 5 is a schematic of an exemplary nitric oxide releasing solution delivery device.

FIG. 6 is a schematic of an exemplary nitric oxide releasing solution delivery device.

FIG. 7, comprising FIGS. 7A through 7D, is a schematic of exemplary nitric oxide releasing solution delivery devices with variable liquid reservoir positions.

FIG. 8 is a schematic of exemplary nitric oxide releasing solution delivery devices with collapsible liquid reservoir.

FIG. 9 is a schematic of an exemplary nitric oxide releasing solution delivery device with a gas cartridge connected to the liquid reservoir.

FIG. 10 is a schematic diagram of the cow nostril model.

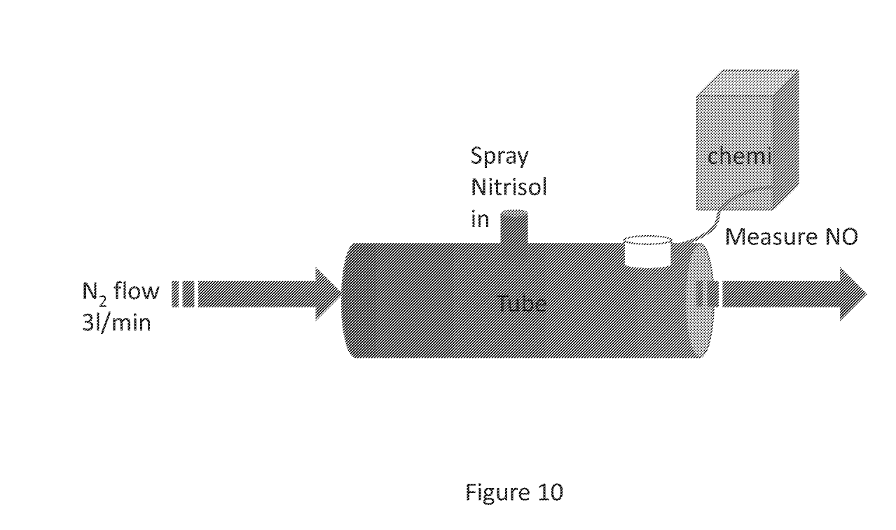

FIG. 11 is a graph of the amount of NO gas present in the system over time.

FIG. 12 is a schematic diagram of the experimental set up.

FIG. 13 is a graph of the shape of the graph of Equation 6.

FIG. 14 is a graph of NO concentration within the chamber over a 30 minute time interval. Circles, squares, and triangles indicate pHs 3.3, 3.4, and 3.5, respectively. Error bars indicate standard deviation.

FIG. 15 is a graph of peak NO concentration plotted against the pH of 100 mM NORS. Regression is an exponential decay relationship where y=8462e^{-1.12x} (R²=0.982). Error bars indicate standard deviation.

FIG. 16 is a graph of the concentration of NO in the chamber over a 30 minute time interval when exposed to NORS at pH 3.45 of varying nitrite concentrations. Circles, squares, triangles, and diamonds indicate 25, 50, 75, and 100 mM nitrites. Error bars indicate standard deviation.

FIG. 17 is a graph of peak NO concentration within the chamber plotted against the concentration of NORS (pH 3.45) placed within the 500 ml nebulizer. Linear regression has equation y=1.61(±0.03)x−1(±2) (R²=0.9987). Error bars indicate standard deviation.

FIG. 18, comprising FIGS. 18A and 18B, is a graph of the release of nitric oxide from a solution as measured by chemiluminescence.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical nitric oxide delivery formulations and delivery systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate. It is to be understood that in the present specification, the use of the term “about” in connection with a numerical value also affords support for the exact numerical value as though it had been recited without the term “about”.

In this specification, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

“NORS” as used herein may refer to a nitric oxide releasing solution or substance.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered.

As used herein, an “instructional material” includes a publication, a recording, a diagram, or any other medium of expression which can be used to communicate the usefulness of a compound, composition, solution, or delivery system of the invention in a kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, solution, or delivery system of the invention or be shipped together with a container which contains the identified compound, composition, solution, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal amenable to the methods described herein. In certain non-limiting embodiments, the patient, subject or individual is a livestock animal, such as cattle, or it may be a human.

Occurrences of the phrase “in one embodiment,” or “in one aspect,” herein do not necessarily all refer to the same embodiment or aspect.

A “therapeutic” treatment is a treatment administered to a subject who exhibits signs and/or symptoms of a disease or disorder, for the purpose of diminishing or eliminating those signs and/or symptoms. Additionally a “therapeutic” treatment may be a treatment administered to a subject who does not exhibit signs and/or symptoms of a disease or disorder, but who is determined to be at risk for development of a given disease or disorder, for the purpose of preventing or delaying onset of such disease or disorder.

As used herein, “treat,” “treatment,” and “treating,” a disease or disorder” means reducing the severity and/or frequency, with which a sign and/or symptom of the disease or disorder is experienced by a subject. The subject may be symptomatic or asymptomatic at the time of treatment. In otherwords, “treat,” “treatment,” or “treating” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

As used herein, an “effective amount” of an agent is an amount sufficient to accomplish a specified task or function desired of the agent. The phrase “therapeutically effective amount,” as used herein, refers to an amount that is sufficient or effective to prevent or treat (delay or prevent the onset of, prevent the progression of, inhibit, decrease or reverse) a disease or disorder in a subject. It is understood that various biological factors may affect the ability of a substance to perform its intended task. Therefore, a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present invention includes a nitric oxide releasing solution capable of reducing the presence of a bacteria, virus or other pathogen in a subject. In one embodiment, the solution may be delivered to at least a portion of the upper respiratory tract of a mammal. In a further embodiment, the solution may be delivered to the nasal passages of cattle to reduce symptoms associated with Bovine Respiratory Disease. In another embodiment, the solution is topically administered to the skin of a mammal, including a human subject, to treat an infected wound.

### NO Releasing Solutions

The formulation of the nitric oxide releasing solution includes the use of a water- or saline-based solution and at least one nitric oxide releasing compound, such as nitrite or a salt thereof. For example, in one embodiment, the solution includes sodium nitrite in a saline solution. Non-limiting examples of nitrites include salts of nitrite such as sodium nitrite, potassium nitrite, barium nitrite, and calcium nitrite, mixed salts of nitrite such as nitrite orotate, and nitrite esters such as amyl nitrite.

Further, the solution of the present invention may be characterized as having a “dormant” state and an “active” state. As contemplated herein, the dormant state of the nitric oxide releasing solution is one in which the pH of the solution is above 4.0 and exhibits a minimal or undetectable production level of nitric oxide gas. In one embodiment, the pH of the dormant state of the nitric oxide releasing solution is between a pH of about 4.0 and a pH of about 7.0. The active state of the nitric oxide releasing solution is one in which the pH of the solution is below 4.0 and exhibits an increased or enhanced production level of nitric oxide gas over an extended period of time. In one embodiment, the pH of the active state of the nitric oxide releasing solution is between a pH of about 1.0 and a pH of about 4.0. In another embodiment, the pH of the active state of the nitric oxide releasing solution is between a pH of about 3.0 and a pH of about 4.0. In one embodiment, the pH is about 3.2. In another embodiment, the pH is about 3.6. Because the nitric oxide releasing solution of the present invention has these two states, the solution can be pre-made, transported and set up for administration while in its dormant state (pH greater than 4.0), without losing any appreciable amount of nitric oxide gas or without losing its ability to produce an effective amount of nitric oxide gas. Then, when a user is ready to deliver or administer the solution for treatment of a subject mammal, the solution can be activated immediately prior to administration to the subject mammal (pH driven below 4.0), thereby maximizing the amount of nitric oxide gas produced by the administered dosage of solution.

In one embodiment, the pH of the solution can be lowered via addition of nitric oxide gas into the solution. Introduction of nitric oxide gas drives the solution reaction towards the reactants, thus reducing the pH (creating more acid), which in turn creates more nitric oxide gas. Accordingly, the solution of the present invention in its active state provides more nitric oxide gas coming out of the solution than is put into the solution.

For example, by introducing sodium nitrite (or other salts of nitrites) to a saline solution it will very slowly produce nitric oxide gas, but in an undetectable amount (as measured by chemiluminescence analysis methodology (ppb sensitivity)). The rate of NO produced from the solution increases as the pH is decreased. Further, it is known that once the pH is below 4.0, then the rate of NO production is significantly increased. An unexpected property of the present invention is that the amount of NO gas coming out of the solution is more than the amount of NO gas added to the solution. NO is produced based on the following equilibrium equations:

NO₂⁻+H⁺→HNO₂ 1.

2HNO₂→N₂O₃+H₂O→H₂O+NO+NO₂ 2a.

3HNO₂ [symbol] 2NO+NO₃⁻+H₂O+H⁺ 2b.

Therefore, acid is needed to donate the H⁺ to the nitrite (NO₂⁻). The more H⁺ present, the faster the reaction will go towards HNO₂ and the more NO will be produced.

As can be seen from these equations, increasing the concentration of nitrites present in the solution (for example 60 mM versus 20 mM), requires more acid to achieve the same pH. In other words, the more HNO₂ produced, the lower the pH will be.

Typically, if sodium nitrite is added to a saline solution, the pH is about 6-7 and minimal NO will be created. However, once NO gas is introduced into the solution, such as by bubbling (minutes), or by rapidly injecting (seconds) NO into the solution in a manner similar to a “carbonation technique,” the pH is rapidly reduced. Accordingly, once the pH is driven below 4.0, the solution is driven to the prolonged production of sufficient NO gas for the effective treatment of the subject.

Interestingly, NO₂⁻ or pH by themselves, even at the optimal levels, are insufficient to have an antimicrobial effect on a subject. However, when these factors are combined, the solution produces enough NO gas to have an antimicrobial effect.

The solution may be administered to the subject as a slow release formulation of NO gas, and optionally with a carrier formulation, such as microspheres, microcapsules, liposomes, etc., as a topical ointment or solution, or in an intranasal injection, as known to one skilled in the art to treat a microbial disease or disorder. By “slow release,” it is meant that an effective amount of NO gas is released from the formulation at a controlled rate such that therapeutically beneficial levels (but below toxic levels) of the component are maintained over an extended period of time ranging from, e.g., about 1 minute to about 24 hours, thus, providing, for example, a 30 to 60 minute, or several hour, dosage form. In a preferred embodiment, the NO gas is released over a period of at least 30 minutes.

### Methods

The present invention provides a method of treating a subject in need comprising the delivery of a nitric oxide releasing solution to a treatment site of the subject. The present method can be used to treat any disease, disorder, or condition where nitric oxide delivery is beneficial. Exemplary diseases, disorders, or conditions, include but are not limited to, respiratory diseases, respiratory infections, wounds, burns, topical infections, inflammatory diseases, and the like. In certain embodiments, the nitric oxide releasing solution is prepared just prior to administration to the subject through the administration of NO or NO₂ containing gas to a dormant solution. For example, as described elsewhere herein, administration of NO or NO₂ containing gas to the dormant solution results in the lowering of the pH of the dormant solution, thereby activating the nitric oxide releasing solution to be administered to the treatment site. In some embodiments, the pH can be lowered by a different mechanisms, such as addition of an acid to the solution. Importantly, the nitric oxide releasing solution provides for extended production of nitric oxide. In one embodiment, the nitric oxide releasing solution produces nitric oxide for a period of between 1 minute and 24 hours. In one embodiment, the nitric oxide releasing solution produces nitric oxide for a period of between 10 and 45 minutes. In one embodiment, the nitric oxide releasing solution produces nitric oxide for at least 15 minutes. In one embodiment, the nitric oxide releasing solution produces nitric oxide for at least 30 minutes. In one embodiment, the nitric oxide releasing solution produces nitric oxide for at least 8 hours. Thus, the administered nitric oxide releasing solution provides for continuous delivery of nitric oxide to the treatment site of the subject. Further, the amount of administered nitric oxide releasing solution may be varied in order to optimize the duration of nitric oxide production and delivery. The nitric oxide releasing solution may be reapplied one or more times, as necessary to effectively treat the subject.

The present invention allows for delivery of nitric oxide to an ambulatory subject. For example, the extended production and delivery of nitric oxide to the treatment site by way of the administered nitric oxide releasing solution allows for the treated subject to remain ambulatory during treatment. Thus, the subject is not constrained to a nitric oxide delivery device during the entire duration of nitric oxide delivery. The present invention provides methods of treatment in any suitable subject, including humans, primates, mammals, cattle, horses, dogs, cats, pigs, sheep, goats, and the like.

In one embodiment, the method comprises the treatment of a respiratory disease in a subject. Exemplary respiratory diseases treated by way of the present method include, but are not limited to emphysema, chronic bronchitis, asthma, adult respiratory syndrome (ARDS), chronic obstructive pulmonary disease (COPD), cystic fibrosis, bovine respiratory disease (BRD), porcine respiratory disease complex (PRDC), and the like. In certain embodiments, the method comprises the treatment of a respiratory disease or disorder caused by a bacterial or viral infection. Treatment of a respiratory disease by way of the present invention comprises the delivery of a nitric oxide releasing solution into the upper respiratory tract of the subject to be treated. For example, in certain embodiments, the nitric oxide releasing solution may be injected, sprayed, inhaled, or instilled into the respiratory tract of the subject. The nitric oxide releasing solution may be administered to the respiratory tract of the subject via the nasal cavity or oral cavity of the subject. In one embodiment, the nitric oxide releasing solution is sprayed into the upper respiratory tract of the subject using the device of the invention, described elsewhere herein. The nitric oxide releasing solution provides for extended nitric oxide production, thereby providing continuous delivery of therapeutic nitric oxide to the upper respiratory tract of the subject.

In one embodiment, the method comprises the treatment of a respiratory infection in a subject, including infections caused by a virus, a fungus, a protozoan, a parasite, an arthropod or a bacterium, including a bacterium that has developed resistance to one or more antibiotics. In certain embodiments, nitric oxide releasing solution, produced by the device of the invention just prior to its use, as described elsewhere herein, is directly administered into the upper respiratory tract of the subject. For example, in one embodiment, the nitric oxide releasing solution is sprayed into the upper respiratory tract of the subject using the device of the invention, as described elsewhere herein. The nitric oxide releasing solution provides for extended nitric oxide production, thereby providing continuous delivery of therapeutic nitric oxide to the upper respiratory infection of the subject.

Patients with open wounds resulting from physical injury or infection or from the result of known diseases such as diabetes or venous stasis disease, have the need to have their wounds treated with a nitric oxide gas or nitric oxide compound. In prior methods, patients being treated with nitric oxide gas are required to remain stationary in a location where the delivery device and high pressure gas source are connected to their wound. As previously described in this application is a method and device for preparing on-site a nitric oxide releasing solution that can provide sustained release.

In one embodiment, the method of the present invention comprises spraying the wound of a subject with a nitric oxide releasing solution that has been prepared just prior to application and then covered with a gas impermeable cover that will retain the produced nitric oxide under the cover and therefore expose the wound to the therapeutic concentration of nitric oxide. The cover may have a small bleed hole to control or limit the pressure under the cover. This allows the subject to be treated and then be ambulatory, eliminating the need for the subject to remain next to the gas source.

In one embodiment, the method comprises the treatment of a wound, including but not limited to, an open wound, cut, scrape, burn, abscess, lesion, surgical wound, trauma wound, disease-associated wound or the like. In certain embodiments, the method comprises administering the dormant solution to the treatment site. In certain embodiments, NO, N₂O, CO₂, or NO₂ containing gas is delivered to the dormant solution which lowers the pH of the dormant solution thereby creating the nitric oxide releasing solution. For example, in one embodiment, the nitric oxide releasing solution is produced by applying NO or NO₂ containing gas to the dormant solution directly on the treatment site. In another embodiment, the nitric oxide solution is produced away from the treatment site, and is then topically applied to the treatment site. In one embodiment, the method comprises administering a gas impermeable cover over the treatment area of the subject, in order to constrain the produced nitric oxide gas over the treatment site. The cover may be applied prior to, during, or after administration of the dormant solution or nitric oxide releasing solution. The nitric oxide releasing solution provides for extended nitric oxide production, thereby providing continuous delivery of therapeutic nitric oxide to the wound of the subject.

In one embodiment, the method comprises the treatment of an infection in a subject, including infections caused by a virus, a fungus, a protozoan, a parasite, an arthropod or a bacterium, including a bacterium that has developed resistance to one or more antibiotics. In certain embodiments, particularly for the treatment of topical infections, the method comprises administering the dormant solution to the treatment site. In certain embodiments, NO or NO₂ containing gas is delivered to the dormant solution which lowers the pH of the dormant solution thereby creating the nitric oxide releasing solution. For example, in one embodiment, the nitric oxide releasing solution is produced by applying NO or NO₂ containing gas to the dormant solution directly on the treatment site. In another embodiment, the nitric oxide solution is produced away from the treatment site, and is then topically applied to the treatment site. In one embodiment, the method comprises administering a gas impermeable cover over the treatment area of the subject, in order to constrain the produced nitric oxide gas over the treatment site. The cover may be applied prior to, during, or after administration of the dormant solution or nitric oxide releasing solution. The nitric oxide releasing solution provides for extended nitric oxide production, thereby providing continuous delivery of therapeutic nitric oxide to the infection of the subject.

In one embodiment, the present invention provides a method of treating skin inflammation, including inflammation associated with psoriasis, dermatitis (atopic, contact, sebborheic, etc), eczema, tinea pedis, and rosacea. In certain embodiments, the method comprises administering the dormant solution to the treatment site. In certain embodiments, NO or NO₂ containing gas is delivered to the dormant solution which lowers the pH of the dormant solution thereby creating the nitric oxide releasing solution. For example, in one embodiment, the nitric oxide releasing solution is produced by applying NO or NO₂ containing gas to the dormant solution directly on the treatment site. In another embodiment, the nitric oxide solution is produced away from the treatment site, and is then topically applied to the treatment site. In one embodiment, the method comprises administering a gas impermeable cover over the treatment area of the subject, in order to constrain the produced nitric oxide gas over the treatment site. The cover may be applied prior to, during, or after administration of the dormant solution or nitric oxide releasing solution. The nitric oxide releasing solution provides for extended nitric oxide production, thereby providing continuous delivery of therapeutic nitric oxide to the treatment site of the subject.

### Delivery Device

The present invention further includes a device for delivering or administering the nitric oxide releasing solution, as described herein. The device allows a user to prepare and deliver the activated solution to a subject, without appreciable loss of nitric oxide gas produced by the formulation prior to administration. Generally, the delivery device includes a spray gun with a fixed volumetric delivery head that delivers a precise volume of liquid per spray. A liquid reservoir is attached to the spray gun and holds the premixed nitric oxide releasing solution in its dormant state. A port on the spray gun enables the attachment of a high pressure gas cartridge containing either NO or NO₂. Once released into the liquid reservoir, the NO or NO₂ activates the solution to produce the dissolved nitric oxide gas, while still contained in the same sealed liquid reservoir. The activated nitric oxide releasing solution is then withdrawn from the reservoir and sprayed onto the treatment site or area. For example, the activated solution may be sprayed into the nostrils of the cattle in brief, measured bursts. The activated solution now lining the nasal passages of the cattle continues to release NO gas for up to 30 minutes, or even longer.

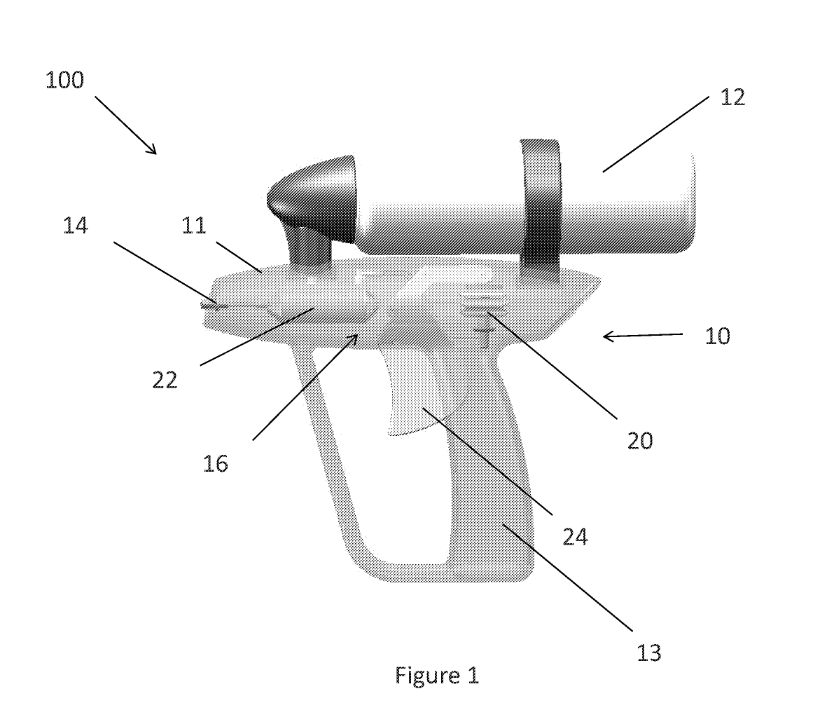

Exemplary devices of the present invention are illustrated in FIGS. 1-9. As shown in FIGS. 1-9, exemplary devices (100, 200, 300, 400, 500, 600, and 700) of the present invention may include a housing 10, a liquid reservoir 12 for storing the nitric oxide releasing solution, a NO or NO₂ gas cartridge 30, a spray nozzle 14, a spray mechanism 16, and a port for introduction of NO or NO₂ gas into liquid reservoir 12.

As contemplated herein, housing 10 may generally resemble a gun, including a barrel 11 and a handle 13 and trigger 24 for ease of use and engagement of spray mechanism 16. Housing 10 may be constructed with standard metals, plastics and other polymers for molding a lightweight and easily maneuverable device of varied design.

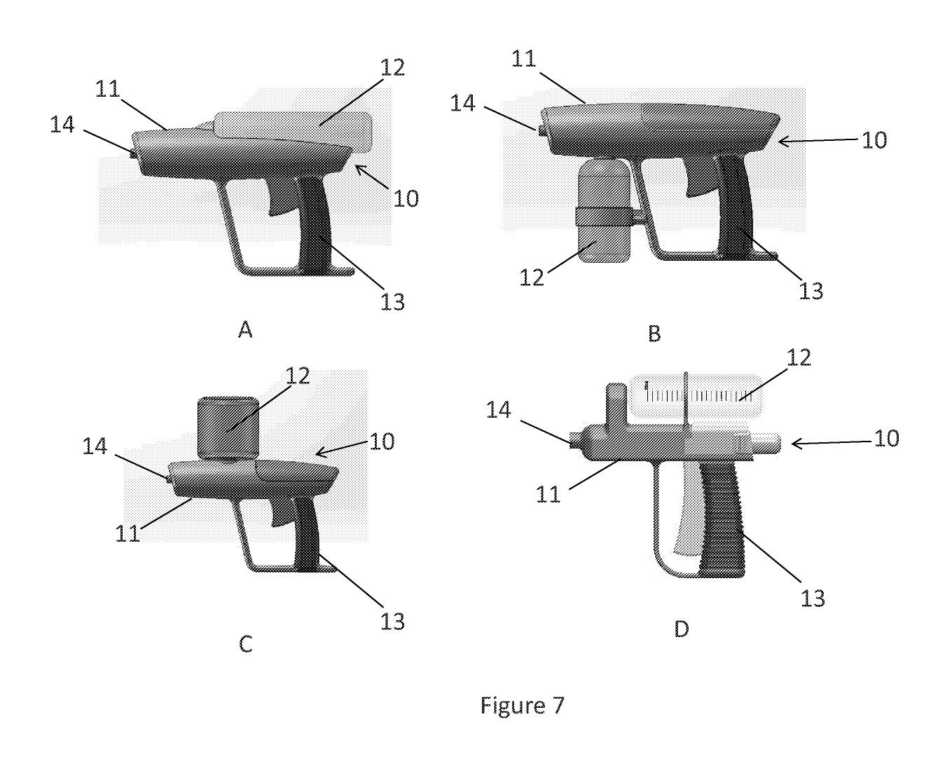

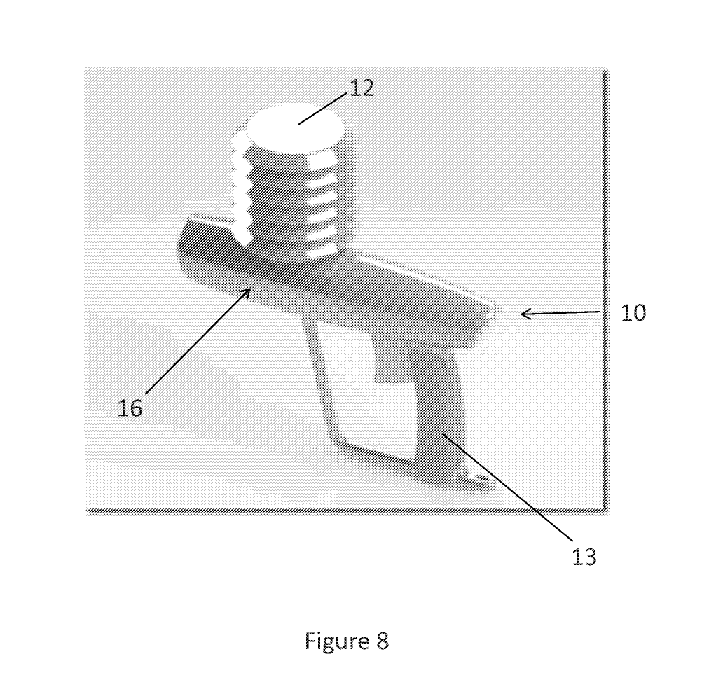

Liquid reservoir 12 may be of any desired size and shape, provided the reservoir is suitable for holding single or multiple doses or application volumes of nitric oxide releasing solution without requiring refill. As shown in FIGS. 7A-7D, liquid reservoir 12 may be positioned relative to the housing 10 in a number of locations, such as, without limitation, partially embedded within the housing barrel 11 (7A), underneath the housing barrel 11 (7B), over top the housing barrel 11 (7C) or suspended above the housing barrel 11 with a raised port connected to the barrel (7D). In another embodiment, as shown in FIG. 8, liquid reservoir 12 may be a collapsible container that enables a substantially zero air volume that is expandable for NO or NO₂ gas injection. In this embodiment, liquid reservoir 12 collapses as the solution is delivered or administered via spray mechanism 16. In certain embodiments, liquid reservoir 12 may be filled with a pre-mixed, recently activated nitric oxide releasing solution, having a pH of below 4.0.

Spray nozzle 14 may be of any standard or desired length, and may initiate any spray pattern known in the art. In some embodiments, spray nozzle 14 may be flexible. In other embodiments, the device may include multiple spray nozzles, such as a dual nostril spray design.

Spray mechanism 16 may operate in a number of ways. Generally, spray mechanism 16 may include spring driven spray technology with manual spring priming and liquid piston filling, pneumatic driven spray technology with manual pneumatic reservoir priming and liquid piston filling, and any other type of manual spray technology as would be understood by those skilled in the art. In still other embodiments, spray mechanism 16 may include a diaphragm pump driven (volumetric) spray technology, or a lead screw spring priming and liquid piston filling, spring driven spray technology. If spray mechanism 16 includes electrical components, a battery pack may be included and integrated within housing 10.

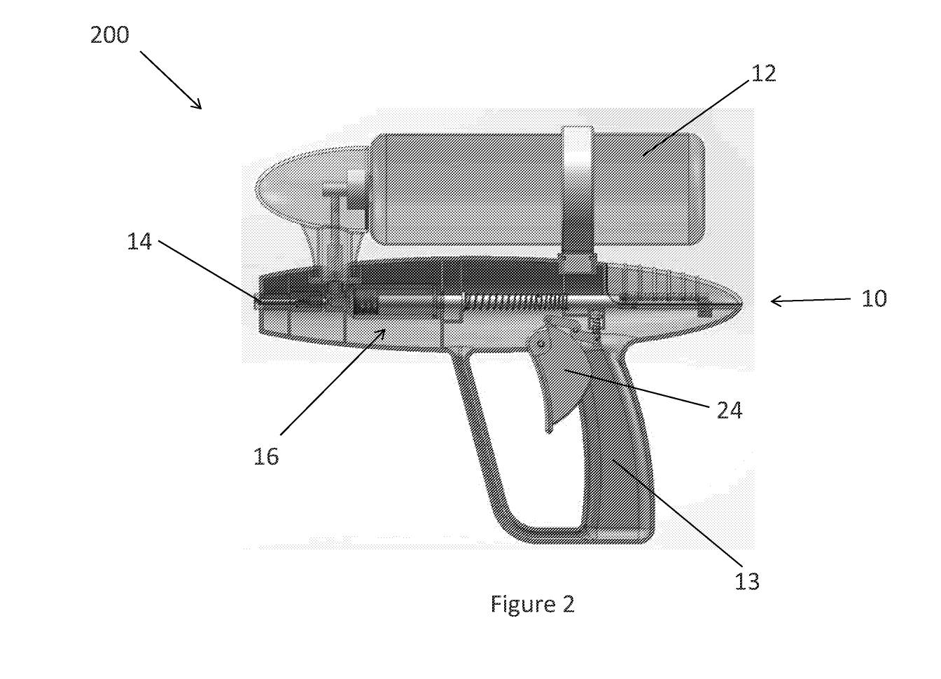

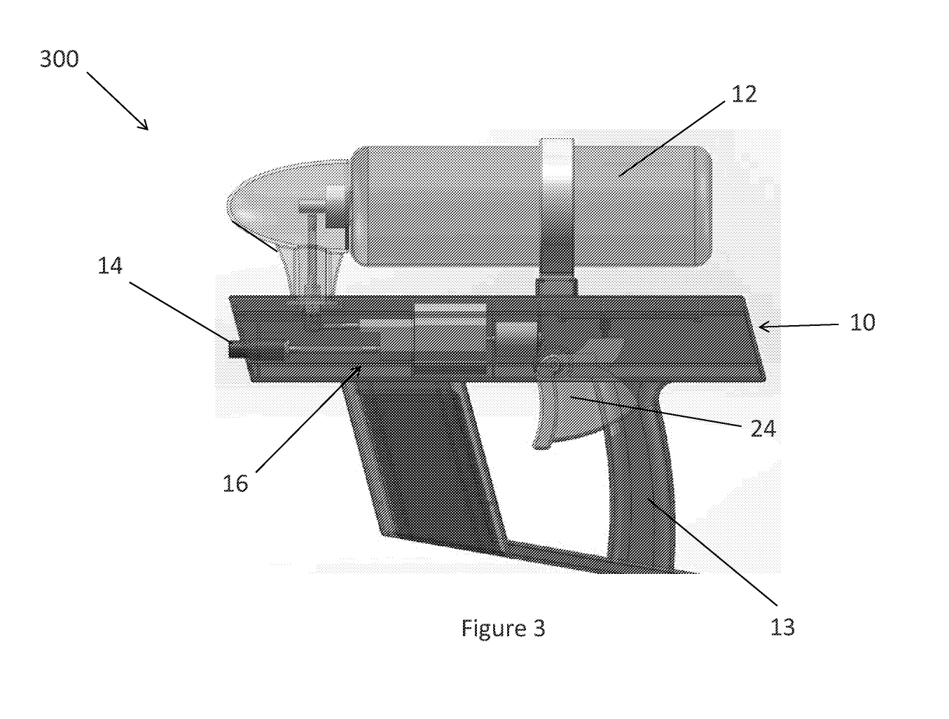

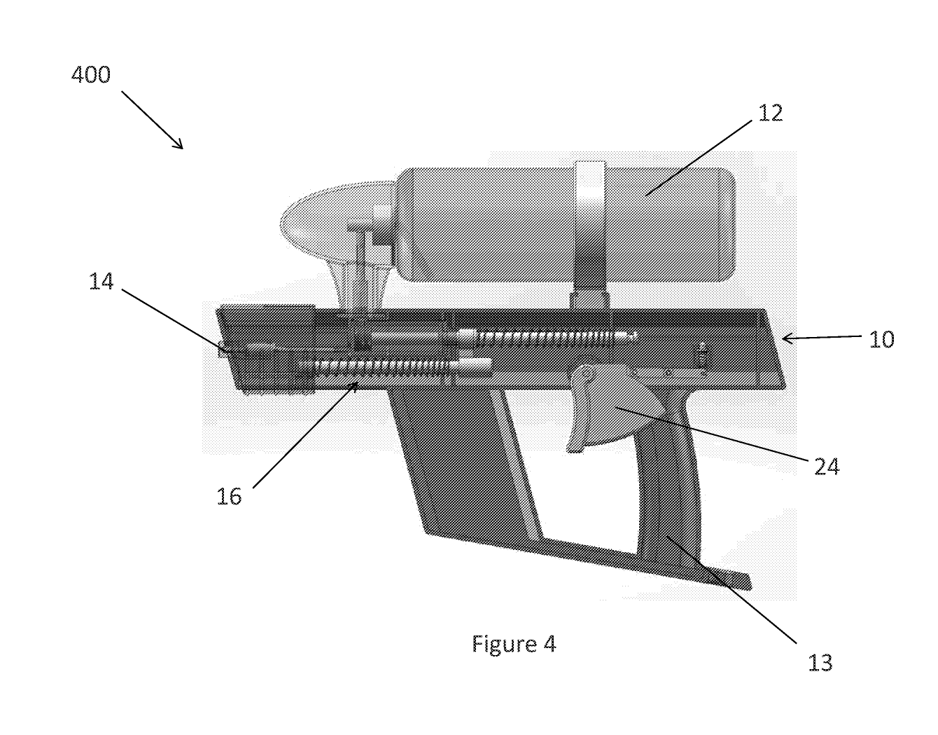

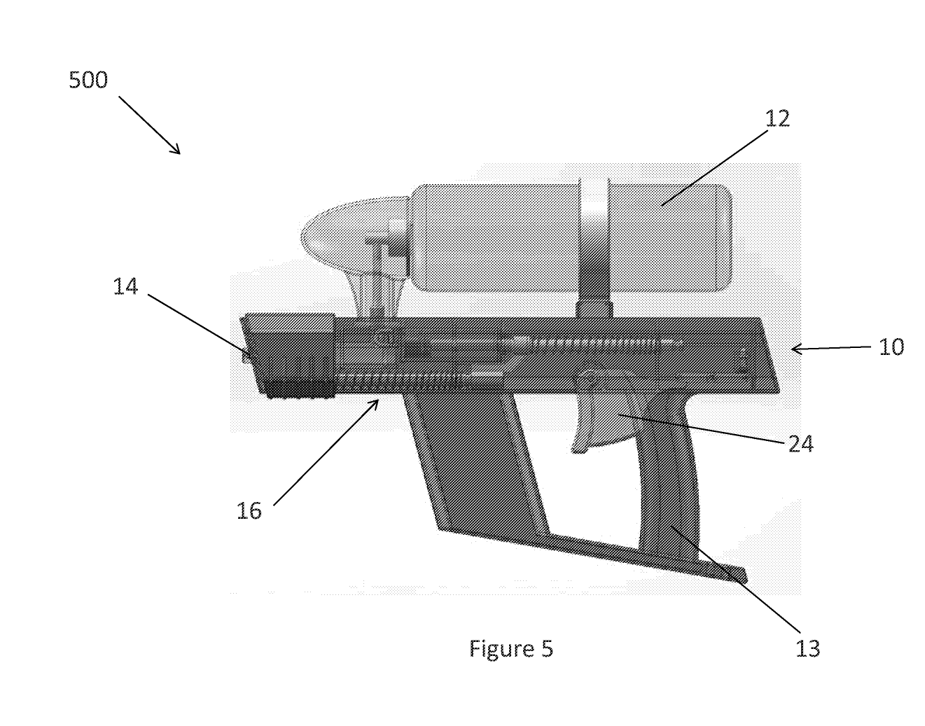

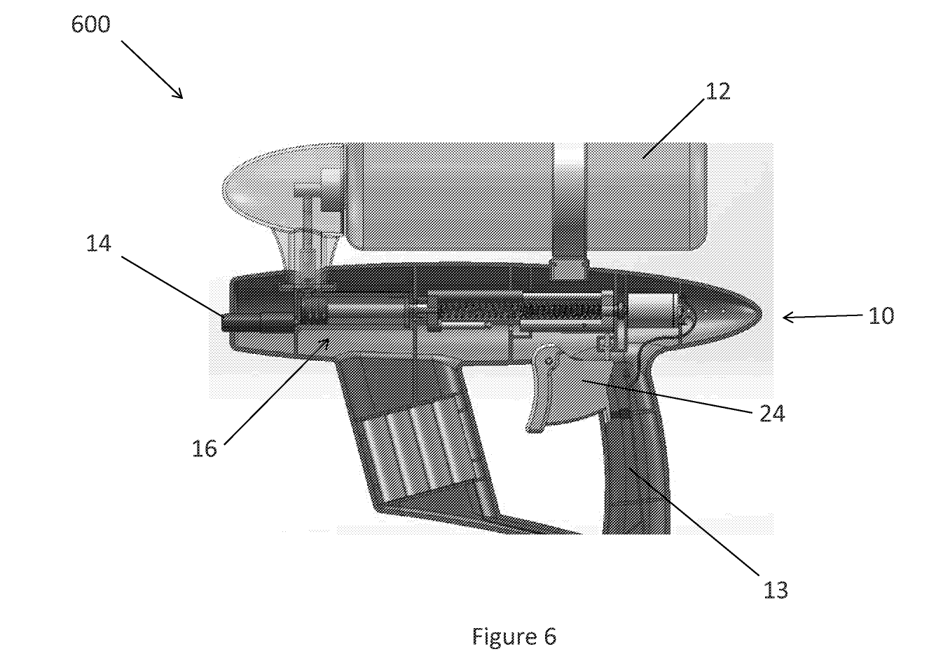

For example, as shown in FIG. 1, the spray mechanism may include spring loaded bellows 20, such that spring loaded bellows 20 will fill a liquid chamber 22 within housing barrel 11 as trigger 24 is released. Then, when trigger 24 is pressed, it will use linkage to push out the pressure of bellows 20, thereby spraying the activated solution through spray nozzle 14. In another example, as shown in FIG. 2, a rear loading lever is pulled back to load the spring and load the nitric oxide releasing solution from reservoir 12 into a piston. Once the spring is engaged, trigger 24 releases the spring force and pushes out the nitric oxide releasing solution to spray nozzle 14. In another example, as shown in FIG. 3, trigger 24 activates a diaphragm pump and a microprocessor may control the nitric oxide releasing solution volume of the spray on each trigger to a predetermined amount, such as 8 ml. In another example, as shown in FIG. 4, a front lever is pulled back and released to fill a liquid piston and load the spring. Then, trigger 24 releases the spring force and plunger to push out the nitric oxide releasing solution to spray nozzle 14. In another example, as shown in FIG. 5, a front lever is pulled back and released to fill a liquid piston and pressurize the air chamber. Then, trigger 24 releases the air chamber air to push out the nitric oxide releasing solution to spray nozzle 14. In another example, as shown in FIG. 6, when trigger 24 is first pressed or another load button is pressed, it will start the motor and the lead screw to load the spring, while loading the liquid piston. A second trigger press will then release the spring force to the plunger and force out the nitric oxide releasing solution to spray nozzle 14.

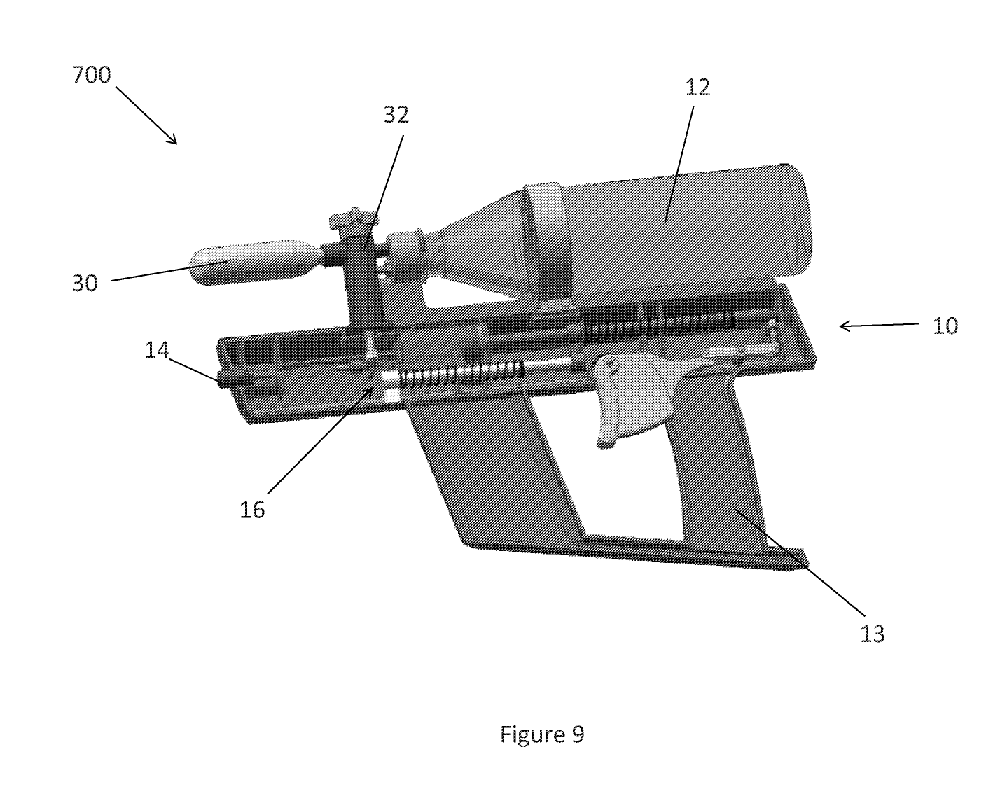

As shown in FIG. 9, in one embodiment, a gas cartridge 30 is connected to liquid reservoir 12 via a valve 32. In such an embodiment, a NO or NO₂ containing gas in gas cartridge 30 can be added to liquid reservoir 12 through valve 32. In another embodiment, NO or NO₂ containing gas can be added directly to liquid reservoir 12 from a gas cartridge without the need for a valve. As described herein, NO or NO₂ containing gas can be delivered to a dormant solution in liquid reservoir 12 to lower the pH of the dormant solution thereby creating a nitric oxide releasing solution.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Production of NO Gas from Solutions of NO₂⁻

The materials and methods employed in these experiments are now described.

Experiments were conducted using 20 Kppm of NO from a pressurized cylinder with a “carbonator connector” system. The following variables were tested:

Containers: 500 ml plastic bottle

Volume: 100 or 200 ml saline;

NO₂⁻ Concentration: 20 mM, 30 mM, or 40 mM level of NO₂⁻.

Driving Pressure: 90 PSI and 30 PSI

Additionally, IV bags of 50 ml with 10 ml of 20 mM, 30 mM, and 40 mM of NO₂⁻ were used.
The results of the experiments are now described.

### Results

Following addition of NO gas to the system, pH change was found to be NO₂⁻ dependent. It was observed that the higher the NO₂⁻ concentration, the less change occurred in pH. Addition of NO to a 20 mM solution of NO₂⁻ resulted in a pH of 3.2. Addition of NO a 30 mM solution NO₂⁻ resulted in a pH of 3.6. Addition of NO to a 40 mM solution of NO resulted in a pH of 4.

It was also observed that the resulting NO₂⁻ concentration increased by about 1 to 10 mM higher after the NO gas injection than baseline. For example, after a rapid injection of 20 Kppm NO gas at 30 psi to a 500 ml bottle containing 100 ml of a 40 mM solution NO₂⁻, a drop in pH to 4 with an increase in NO₂⁻ concentration to 50 mM concentration was observed.

When enough NO₂⁻ is present in the solution, the NO will drive pH down, thereby creating more NO gas from the NO₂⁻. NO gas is driven into the aqueous solution, immediately increasing the concentration of NO₂⁻ and H⁺, which ultimately shifts the equilibrium toward more NO production and release. Production of NO is therefore faster since more NO₂⁻ is present in the system and the pH is lower. If 100 ppm of NO gas is added into the solution, 100 ppm of NO gas can be produced through reaction of NO₂⁻ in the solution, and not from the gas itself. Although not wishing to be bound by any particular theory, this may be due to transmutation from the gas phase to a liquid intermediate, and then back to gas phase.

Measuring NO Gas that is Released from 60 mM NO₂⁻ Solutions and Injected into a “Cow Nostril Model”

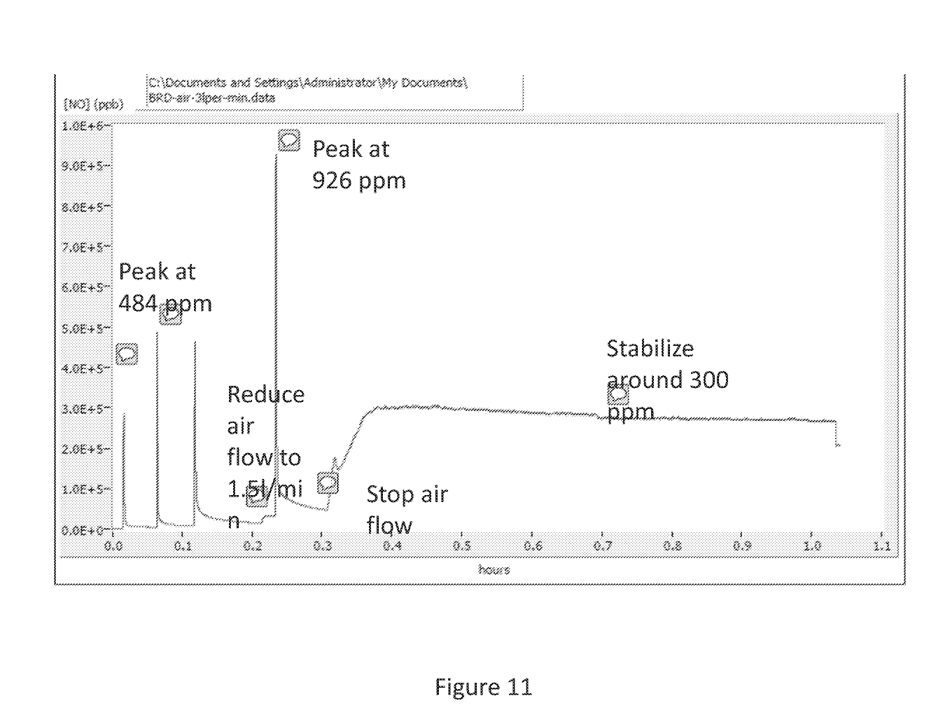

In the model system (FIG. 11), 16 ml of a 60 mM solution of at a constant pressure was sprayed in each nostril (tube) twice (64 ml total solution used). Airflow was then set to 3 l/min (to imitate breathing), reduced to 1.5 ml, and then stopped to accumulate a high concentration of NO. NO was measured constantly throughout using a chemiluminescence.

Each spray into the tube resulted in a peak on NO gas (FIG. 11). The highest peak was 926 ppm and each peak was reduced within 10 seconds to the baseline. Following each peak, baseline became a bit higher. When airflow was stopped, the NO measured stabilized on 300 ppm for the time measured (an hour). This amount was reached after spraying 64 ml of 60 mM NORS.

### Example 2

### Slow Release of Nitric Oxide Gas from Solution of Sodium Nitrite and Citric Acid Monohydrate

The materials and methods employed in these experiments are now described.

### Nitric Oxide Release

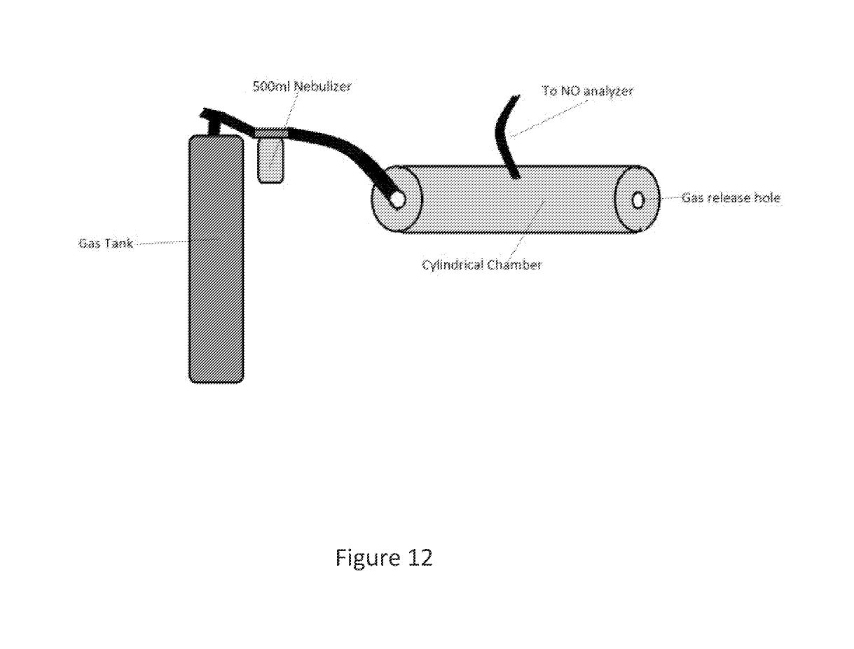

250 ml of distilled water (dH₂O) was added to a 500 ml Erlenmeyer flask. A predetermined mass of solid sodium nitrite (NaNO₂) and citric acid monohydrate were then added to the Erlenmeyer flask and the solution was rapidly mixed to completely dissolve the solids. NORS of 100 mM nitrites at pHs 3.3, 3.4, and 3.5 as well as 25, 50, 75, and 100 mM nitrites at pH 3.45 were tested. pH was measured 1 minute after solids dissolved to ensure the liquid was homogenous and to avoid irregularities created by the initial burst of gases. After pH was confirmed to be within ±0.03 pH units of the desired pH, 250 ml of this solution was added to a clean 500 ml nebulizer which was immediately screwed onto its fitting attached to a compressed air tank and connected to a plastic cylindrical chamber (FIG. 12).

Compressed air was pasted through the nebulizer at a rate of 10 L/min. NO concentration was measured every 15, 60, and 300 seconds between 0-4, 4-10, and 10-30 minutes, respectively, and was facilitated via the use of chemiluminescence measured by a Sievers Nitric Oxide Analyzer calibrated using NO standards and set to draw 250 ml of sample air per minute. After 30 minutes, gas flow was stopped and the pH and volume of the remaining solution in the 500 mL nebulizer were measured. pH change and volume of solution used over 30 minutes was subsequently calculated.

### Nitrogen Dioxide Release

Nitrogen Dioxide levels were measured using a NO/NO₂ analyzer after 5 and 30 minutes of nebulizer use.

### Analysis of Data

It was hypothesized that the rate of NO production from a solution of acidified nitrites was first order in nitrite and acid concentration as given by equation 1, where k′ is the rate constant:

$${( \frac{d [ NO ]}{d t} )}_{sol ′ n} = {k}^{′} [ {NO}_{2}^{-} ] [ {H}_{3} {O}^{+} ] \qquad ( Equation 1 )$$

Equation 3 relates the rate of nitric oxide production to pH and nitrites using Equation 2:

$$\begin{matrix}[ {H}_{3} {O}^{+} ] = {10}^{- pH} & ( Equation 2 ) \\ {( \frac{d [ NO ]}{d t} )}_{sol ′ n} = {k}^{′} [ {NO}_{2}^{-} ] {10}^{- pH} & ( Equation 3 )\end{matrix}$$

Therefore, it was hypothesized that a linear relationship between nitrite concentration and rate of NO production at constant pH would be observed, as well as an exponential decay relationship between pH and the rate of NO production at constant nitrite concentration. These analyses were completed using plots of NORS at 100 mM nitrites, pH 3.3, 3.4, and 3.5 as well as 25, 50, 75, and 100 mM nitrites at pH 3.45.

The rate of NO production was measured as follows. As depicted by FIG. 12, gases are able to enter and leave the cylindrical chamber. The concentration of NO can be related to time using equation 4:

$$\frac{d [ NO ]}{d t} = {k}_{in} - {k}_{out} = k - [ NO ] R \qquad ( Equation 4 )$$

where k is the rate of NO release from NORS (in mol/min) and R is the rate of gas leaving the chamber (in L/min). Solving this differential equation and assuming the initial concentration of NO in the chamber is zero results in equation 5, where [NO](t) is the concentration of NO at time, t:

$$[ NO ] ( t ) = \frac{k}{R} ( 1 - {e}^{- Rt} ) \qquad ( Equation 5 )$$

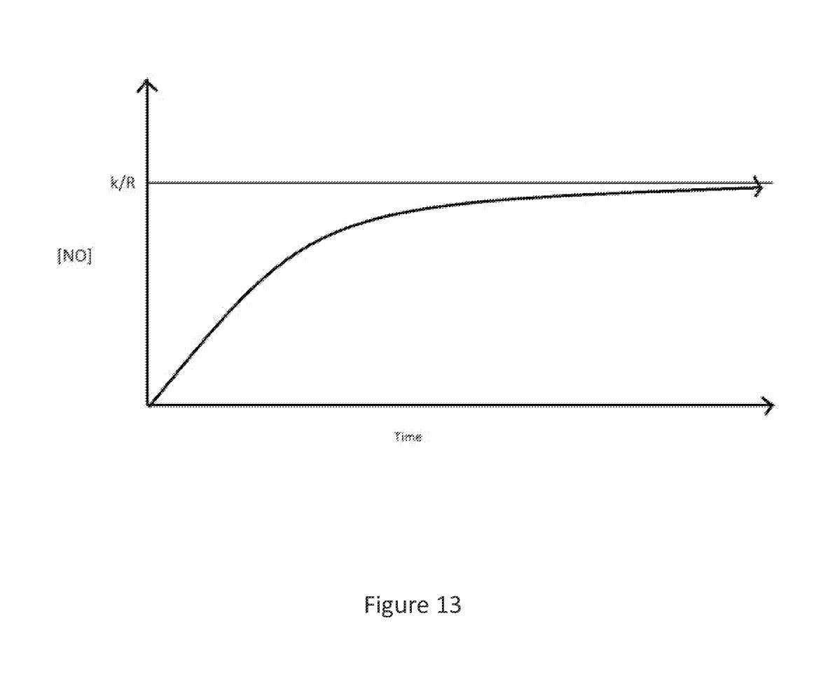

This relationship has the shape depicted by FIG. 13.
As depicted in Equation 6:

$${lim}_{t -> ∞} [ NO ] ( t ) = \frac{k}{R} = {[ NO ]}_{ss} \qquad ( Equation 6 )$$

where [NO]ₛₛ is the steady state concentration of NO (A constant concentration of NO in the chamber).

Therefore, the rate of NO production from the NORS, k, is directly proportional to the steady state concentration measured within the chamber and the rate of gas loss, which must be equal to 10 L/min in order to prevent the chamber from being pressurized. By measuring [NO]ₛₛ the rate of NO production from the NORS in the nebulizer can be predicted using Equation 7:

$${( \frac{d [ NO ]}{d t} )}_{sol ′ n} = k = {R [ NO ]}_{ss} = {k}^{′} [ {NO}_{2}^{-} ] {10}^{- pH} \qquad ( Equation 7 )$$

This relationship assumes k to be constant throughout the experiment, however, k would be expected to decrease after reaching a peak rate due to loss of nitrites and a rise in pH. As such, [NO]ₛₛ is assumed to be equal to the peak [NO].
The results of the experiments are now described.

### Nitric Oxide Release

250 ml of NORS at varying concentrations and pHs was placed within a 500 ml nebulizer and subjected to an air flow of 10 L/min. The rate of NO production from the NORS was measured indirectly through measuring the steady state concentration of NO within the chamber illustrated by FIG. 12.

### Impact of pH on the Rate of NO Production:

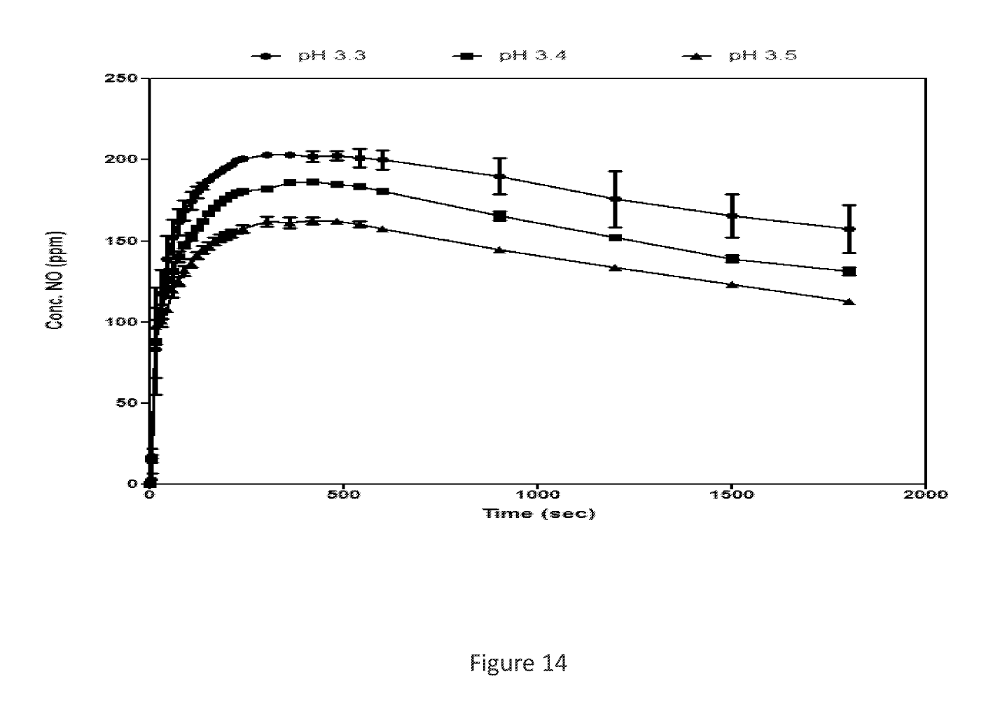

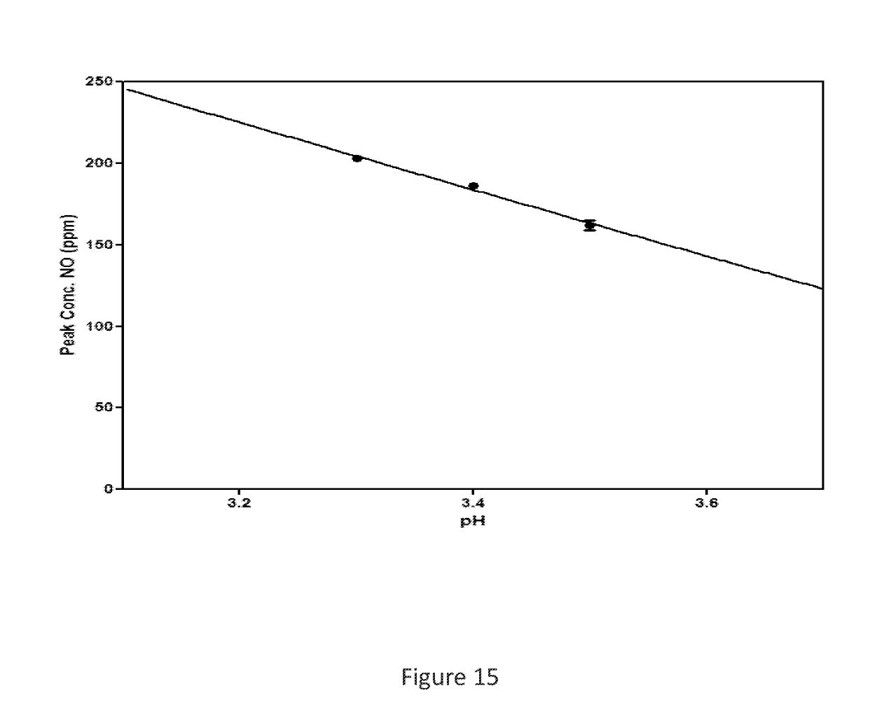

FIG. 14 depicts the change in NO concentration, as measured by chemiluminescence, within the chamber over a 30 minute time period. As depicted, three 100 mM NORS solutions of pHs 3.3, 3.4, and 3.5 were tested. All three pH values rendered an initial rapid increase in NO concentration which subsided as time progressed. All pHs reached a peak [NO] at 7 minutes, with the peak [NO] being inversely related to the pH. pH 3.3, 3.4, and 3.5 gave peak [NO] equal to 203(±1), 186(±1), and 167(±5) ppm, respectively. NO concentration at 30 minutes was roughly 75% of the peak [NO]. FIG. 15 shows a plot of the measured peak [NO] against the pH of the 100 mM NORS. As illustrated, peak [NO] appeared to be linearly related to pH, however upon regressional analysis, the relationship was best fit with an exponential decay equation (R²=0.982 and 0.980 for an exponential and linear regression, respectively). pH rose an average of 0.22 units over the 30 minutes.

### Impact of Nitrite Concentration on the Rate of NO Production:

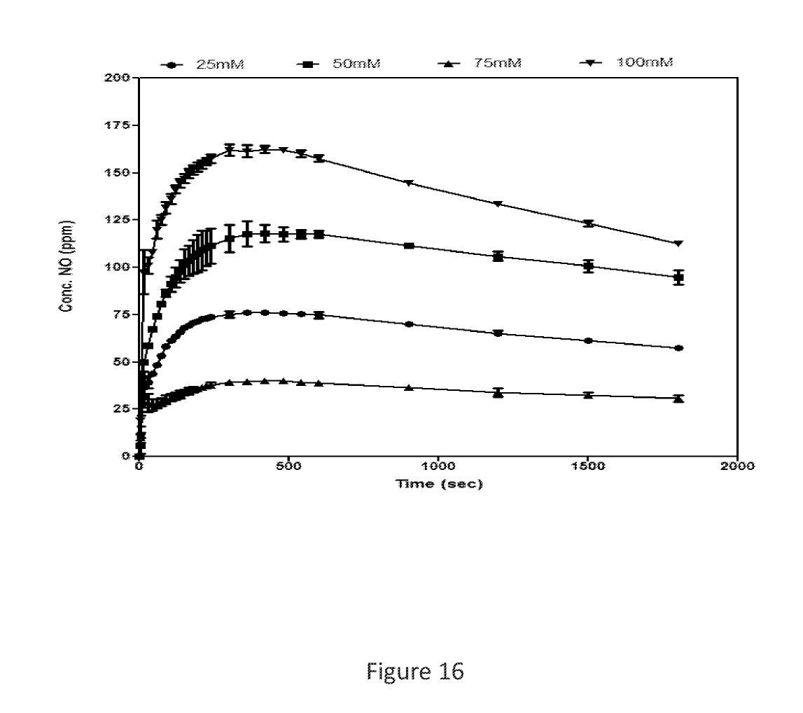

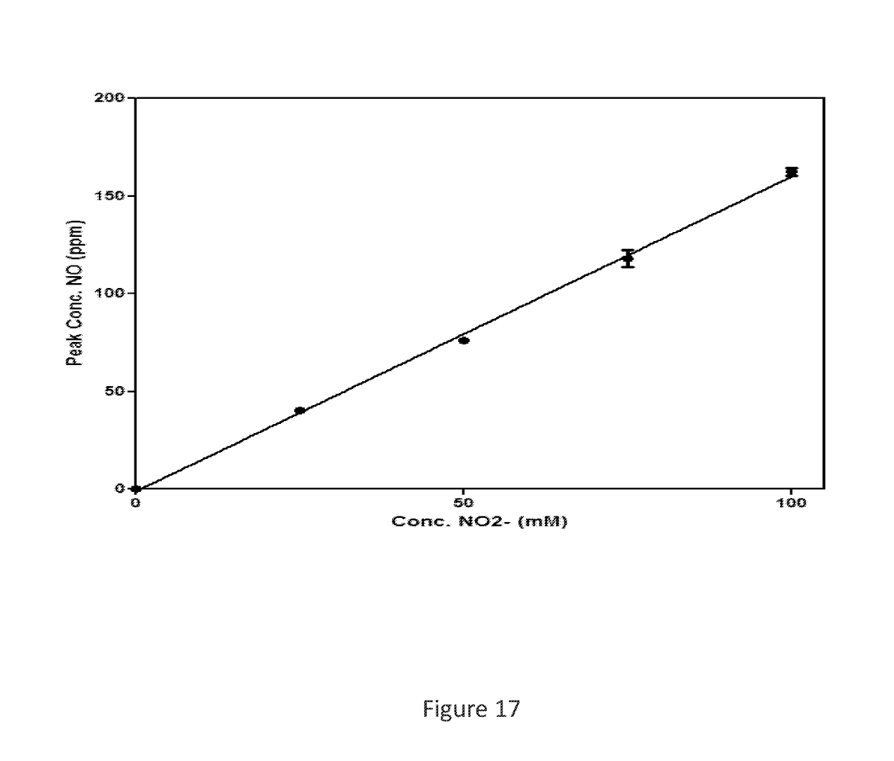

FIG. 16 depicts the change in NO concentration within the chamber over a 30 minute time period when using pH 3.45 NORS of nitrite concentrations 25, 50, 75, and 100 mM. As illustrated, all four nitrite concentrations rendered an initial rapid increase in the concentration of NO within the chamber that decreased over time. Peak NO concentrations occurred roughly 7 minutes after start of the experiment for all concentrations. All four concentrations of NORS saw a slow reduction in NO concentration following their peak values at 7 minutes. 25, 50, 75, and 100 mM NORS (pH 3.45) gave peak [NO] of 39(±1), 76(±1), 118(±5), and 162(±2) ppm, respectively. The [NO] at 30 minutes was roughly 75% of the peak [NO]. Peak NO concentration is plotted against nitrite concentration in FIG. 17. As depicted, peak NO concentration correlated linearly with nitrite concentration (R²=0.9987).

It was observed that the peak nitric oxide concentration is directly proportional to nitrite concentration and exponentially related to pH. These observations corroborate the initial hypothesis of Equation 3. If Equation 3 is assumed to be a correct representation of the rate on NO production, then use equation 7 relating the steady state NO concentration to the nitrite concentration and pH to determine the rate constant, k′, of this reaction:

$$\frac{{R [ NO ]}_{ss}}{[ {NO}_{2}^{-} ] {10}^{- pH}} = {k}^{′} \qquad ( Equation 8 )$$

Table 1 shows the calculated k′ values from each NORS solution tested and provides an average value for k′ plus an estimate of its error. [NO]ₛₛ, measured in ppm, is converted to mM using Equations 9, 10, and 11.

$$\begin{matrix}x ppm NO = \frac{x mol NO}{{10}^{6} mol air} & ( Equation 9 ) \\ \begin{matrix}V = \frac{nRT}{p} \\ = \frac{{10}^{6} ( mol air ) * 00321 ( L aim {mol}^{- 1} {K}^{- 1} ) * 298 ( K )}{1 ( aim )} \\ = 2.45 \times {10}^{7} L\end{matrix} & ( Equation 10 ) \\ x mM NO = \frac{x ppm NO}{2.45 \times {10}^{7} L} \times \frac{1000 mM}{M} & ( Equation 11 )\end{matrix}$$

TABLE 1
Determination of the rate constant, k′, for the production of NO from NORS.

| | | | δ[NO]ₛₛ | | | | |
|---|---|---|---|---|---|---|---|
| [NO₂⁻] (mM) | pH | [NO]ₛₛ (ppm) | (ppm) | [NO]ₛₛ (mM) | δ[NO]ₛₛ (mM) | k′ (min⁻¹) | δk′ (min⁻¹) |
| 100 | 3.3 | 203.1 | 1.3 | 0.008290 | 5E−05 | 1.654 | 0.010 |
| 100 | 3.4 | 186.5 | 0.1 | 0.007612 | 4E−06 | 1.912 | 0.001 |
| 100 | 3.5 | 162.1 | 3.1 | 0.006616 | 1E−4 | 2.092 | 0.040 |
| 25 | 3.45 | 40.2 | 0.2 | 0.001639 | 8E−06 | 1.850 | 0.009 |
| 50 | 3.45 | 76.3 | 1.2 | 0.003112 | 5E−05 | 1.754 | 0.027 |
| 75 | 3.45 | 118.0 | 4.5 | 0.004816 | 2E−4 | 1.810 | 0.069 |

Therefore, the rate of NO production from NORS between 25 and 100 mM nitrites and pH 3.2 to 3.7 is given by equation 12 where d[NO]/dt is in mM/min and [NO₂⁻] is in mM:

$$\frac{d [ NO ]}{d t} = 1.85 ( \pm 0.03 ) [ {NO}_{2}^{-} ] {10}^{- pH} \qquad ( Equation 12 )$$

These results support the hypothesis that NORS, which can be prepared in seconds from pre-weighed reagents, can be useful in a clinical application. There are also some advantages in using a NORS and nebulizer system as opposed to a direct gas method. For example, over the 30 minute experiment, 12(±2) ml of NORS was removed from the 500 ml nebulizer, indicating that it had been converted into water droplets deposited inside the chamber. These condensed water droplets contained high levels of nitrites (>50 mM). This result supports the hypothesis that they could continue to release NO directly into their surroundings. pH levels of the condensed water could not be ascertained. Although not wishing to be bound by any particular theory, this result suggests that a lower nitrite concentration could be used since a 20 mM NORS (pH 3.7) has been found to be sufficient to be completely fungicidal and bactericidal. Additionally, water droplets deposited within the upper and lower airways of the lungs could continuously provide a dose of NO to infecting pathogens.

### Example 3

### Measurement of NO Gas Released from a 40 mM NO₂⁻ Solution

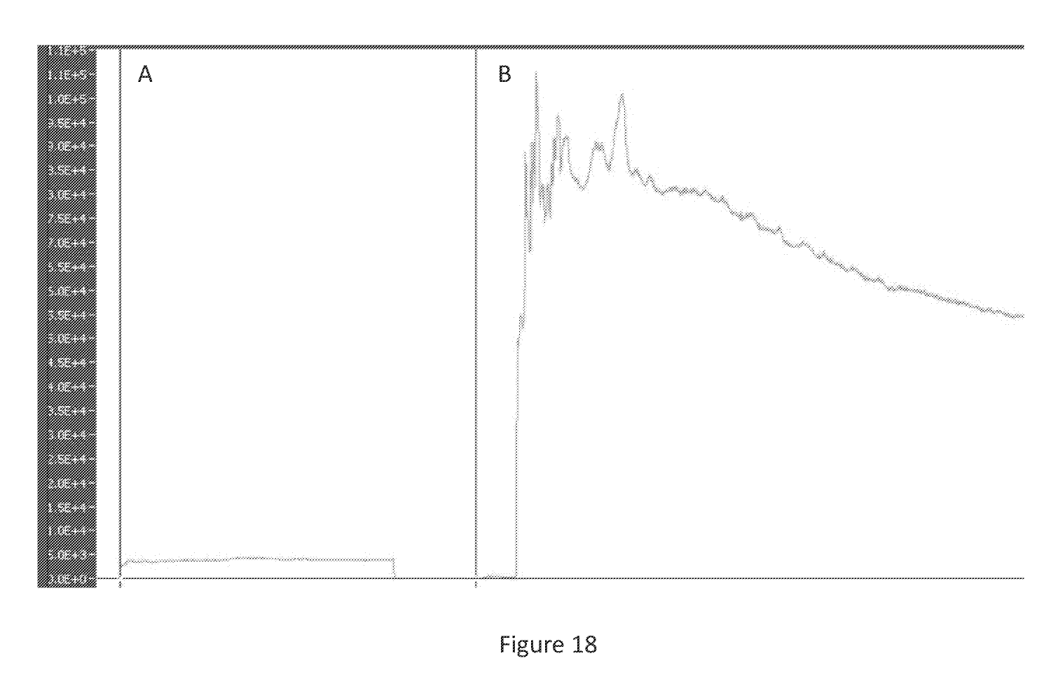

FIG. 18 shows the amount of NO gas released from a 40 mM NO₂⁻ solution, as measured by chemiluminescence. A 40 mM NaNO₂ solution with a pH of 4.5 was placed in a bottle. As shown in FIG. 18A, NO gas released from this solution was constant at a measured value of about 5 ppm. NO gas was then injected into the solution, and the solution was transferred to a new bottle. NO gas released from the solution after NO gas injection was measured, as shown in FIG. 18B, at a peak value of about 110 ppm, before leveling off to about 55 ppm. The pH of the solution after injection of NO gas was measured at 3.56.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of activating a nitric oxide releasing solution, comprising the steps of:
providing a liquid solution that includes at least one nitrite or salt thereof with a pH of greater than 4.0, and contacting the solution with nitric oxide or nitrogen dioxide or other acidifying gas, thereby reducing the pH of the solution to below 4.0, such that the solution releases nitric oxide gas at an increased rate.

2. The method of claim 1, wherein the at least one nitrite or salt thereof is sodium nitrite.

3. The method of claim 1, wherein the liquid solution is a saline solution.

4. The method of claim 1, wherein the increased release rate of nitric oxide gas runs between 1-60 minutes.

5. The method of claim 1, wherein the increased release rate of nitric oxide gas runs at least 30 minutes.

6. The method of claim 1, wherein the pH of the solution is between 3.2 and 3.9 after contact with the nitric oxide or nitrogen dioxide gas.

7. The method of claim 6, wherein the pH is about 3.6 after contact with the nitric oxide or nitrogen dioxide gas.

8. A device for delivering a nitric oxide releasing solution to a subject, comprising:
a housing;
a solution reservoir for holding a liquid nitric oxide releasing solution;
a container of nitric oxide or nitrogen dioxide gas;
at least one spray nozzle; and
a spray mechanism within the housing.

9. The device of claim 8, wherein nitric oxide or nitrogen dioxide gas from the container is injected into the liquid nitric oxide releasing solution, and the spray mechanism draws a volume of liquid solution from the solution reservoir and sprays the volume of solution through the at least one spray nozzle.

10. The device of claim 8, wherein the solution reservoir is collapsible.

11. The device of claim 8, wherein the spray mechanism comprises spring loaded bellows to draw an amount of liquid solution from the solution reservoir and push the liquid solution through the at least one spray nozzle.

12. The device of claim 8 wherein the spray mechanism comprises a piston, wherein an amount of liquid solution from the solution reservoir is drawn into the piston and subsequently pushed out through the at least one spray nozzle.

13. The device of claim 8, wherein the spray mechanism comprises a diaphragm pump and a microprocessor.

14. The device of claim 13, further comprising an electrical power source.

15. The device of claim 14, wherein the microprocessor controls the nitric oxide releasing solution volume of the spray to a predetermined amount.

16. The device of claim 14, further comprising a motor.

17. A method of administering nitric oxide to a subject in need thereof, comprising:
providing a solution that includes at least one nitrite or salt thereof in a liquid solution having a pH of greater than 4.0;
contacting the solution with nitric oxide or nitrogen dioxide gas, thereby reducing the pH of the solution to below 4.0; and
delivering the liquid solution to a treatment site of the subject.

18. The method of claim 17, wherein the administration of nitric oxide is for treating a respiratory disease or disorder in the subject.

19. The method of claim 18, wherein the respiratory disease or disorder is one selected from the group consisting of emphysema, chronic bronchitis, asthma, adult respiratory syndrome (ARDS), chronic obstructive pulmonary disease (COPD), cystic fibrosis, bovine respiratory disease (BRD), and porcine respiratory disease complex (PRDC).

20. The method of claim 18, wherein the respiratory disease or disorder is caused by an infection of a virus, a fungus, a protozoan, a parasite, an arthropod, a bacterium, or a bacterium that has developed resistance to one or more antibiotics.

21. The method of claim 17, wherein the treatment site is a wound of the subject.

22. The method of claim 21, further comprising covering the wound with a gas impermeable cover.

23. The method of claim 22, wherein the cover comprises at least one bleed hole to control or limit pressure between the cover and the treatment site.

24. The method of claim 22, wherein the wound is an open wound, a cut, a scrape, a burn, an abscess, a lesion, a surgical wound, a trauma wound, tinea pedis, or a disease-associated wound.
